(19) United States
(12) Patent Application Publication
(10) Pub. No.: US 2024/0180406 A1
(43) Pub. Date: Jun. 6, 2024

(54) ADJUSTABLE ACTUATION MECHANISMS FOR ENDOSCOPES

(71) Applicant: GYRUS ACMI, INC. D/B/A OLYMPUS SURGICAL TECHNOLOGIES AMERICA, Westborough, MA (US)

(72) Inventor: Kester Julian Batchelor, Mound, MN (US)

(21) Appl. No.: 18/523,398

(22) Filed: Nov. 29, 2023

Related U.S. Application Data
(60) Provisional application No. 63/385,841, filed on Dec. 2, 2022.

(51) Int. Cl. A61B 1/005; A61B 1/00

(52) U.S. Cl. CPC A61B 1/0057; A61B 1/00006; A61B 1/00148; A61B 1/0053

(57) ABSTRACT

A controller for an endoscope comprises a handle, a working channel opening connected to the handle at a first location, a control actuator connected to the handle at a second location, a first pull wire extending from the control actuator to the working channel opening within the handle, and a first pull wire tensioner through which the first pull wire extends, wherein the first pull wire tensioner is configured to remove slack from the first pull wire between the first location and the second location.

### PRIORITY CLAIM

This application claims the benefit of priority to U.S. Provisional Patent Application Ser. No. 63/385,841, filed Dec. 2, 2022, the contents of which are incorporated herein by reference in their entirety.

### TECHNICAL FIELD

The present disclosure relates generally to medical devices comprising elongate bodies, such as an endoscope, configured to be inserted into incisions or openings in anatomy of a patient to provide diagnostic or treatment operations.

More specifically, the present disclosure relates to control devices that can be attached to a proximal portion of an elongate body to control or position diagnostic or treatment devices attached to a distal portion of the elongate body.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include the gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract, lungs), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as a biopsy device, catheter, ablation device and the like. Such systems can be helpful in guiding endoscopes to anatomic locations within the body that are difficult to reach. For example, some anatomic locations can only be accessed with an endoscope after insertion through a circuitous path. Control of the endoscope to reach the anatomic location is performed by a control device attached to a proximal portion of the device. Sometimes it can be required to control both the insertion scope and the therapeutic device form separate control devices. For example, sometimes it can be desirable to hold a position of the distal end of the endoscope with a controller for the endoscope while operating the therapeutic device inserted in the endoscope with a separate controller device.

In additional examples, duodenoscopy procedures (e.g., Endoscopic Retrograde Cholangio-Pancreatography, hereinafter “ERCP” procedures) involve the use of an auxiliary scope (also referred to as a daughter scope or cholangioscope) that can be advanced through the working channel of a main scope (also referred to as a mother scope or duodenoscope). Furthermore, another device, such as a tissue retrieval device used for biopsies, can be inserted into the auxiliary scope. As such, it can be required to control the mains scope, auxiliary scope and therapeutic device with three separate control devices.

In view of the foregoing, there is a continuous need to improve the operatbility of control devices for main scopes, auxiliary scopes and therapeutic devices.

### SUMMARY

The present inventor has recognized that problems to be solved with conventional medical devices include, among other things, 1) the difficulty of operating multiple actuators, e.g., buttons and levers, on a controller. For example, some actuators are positioned to be accessible to fingers for one-handed use, which can result in all of the actuators being difficult to reach for some users, such as those with smaller hands. Additionally, some controllers are configured for two-handed use, which can sometimes result in awkward positioning of one hand relative to the other hand, which can lead to muscle strain after prolonged use. Furthermore, in other scenarios, a controller for an auxiliary scope or therapeutic device is sometimes attached to a controller for a main scope, such as when an auxiliary scope or therapeutic device is introduced into the working channel of the main scope. This coupling of the auxiliary controller to the main controller can result in awkward positioning of the hands of a user.

The present disclosure can provide solutions to these and other problems by providing systems, devices and methods relating to ergonomic controls for endoscopes and other scopes. In particular, the present disclosure provides scope controllers having adjustably positionable controller components or control actuators. As such, the position between two actuators or the position between actuators and a coupling feature for an auxiliary scope can be adjusted to provide more ergonomic and user-friendly positioning.

Furthermore, the present inventor has recognized that there is difficulty in producing adjustable controllers for scopes due to, for example, the presence of pull wires within the controller. In order for pull wires to operate, it is desirable for slack within the pull wires to be eliminated. Slack within pull wires introduces a lack of responsiveness in the control actuators. As such, it is typically required that control actuators for pull wires be axially aligned or have line-of-sight with entrances into the working channel of the scope so that proper tension can be applied to the pull wires.

The present disclosure can provide solutions to these and other problems by providing systems, devices and methods relating to pull wires that can be tensioned without line-of-sight between the control actuators and the working channel. As such, the handpiece for the scope controller can separate the control actuators and the working channel into offset handpiece components, moveable handpiece components or separated handpiece components.

In an example, a controller for an endoscope comprises a handle, a working channel opening connected to the handle at a first location, a control actuator connected to the handle at a second location, a first pull wire extending from the control actuator to the working channel opening within the handle, and a first pull wire tensioner through which the first pull wire extends, wherein the first pull wire tensioner is configured to remove slack from the first pull wire between the first location and the second location.

### BRIEF DESCRIPTION OF DRAWINGS

FIG. 1 is a schematic view of a scope having a controller to which is connected a catheter.

FIG. 2 is a schematic cross-sectional view of a handle for the catheter of FIG. 1 showing an elongate catheter body extending therethrough.

FIG. 3 is a schematic view of a distal portion of the catheter of FIG. 2.

FIG. 4 is a schematic cross-sectional view of a distal portion of the catheter of FIGS. 2 and 3.

FIG. 5 is a schematic block diagram of electronic systems configured for use with the scope and catheter of FIGS. 1-4.

FIG. 6 is side view of the controller of the scope of FIG. 1 showing pull wires for controlling a distal tip of the scope.

FIG. 7 is a perspective view of the distal tip of the scope of FIG. 1 showing articulation of the distal tip.

FIG. 8A is perspective view of an adjustable controller of the present disclosure in a retracted state.

FIG. 8B is a perspective view of the adjustable controller of FIG. 8A in an extended state.

FIG. 9A is a partially broken-away perspective view of the adjustable controller of FIG. 8B showing slide components to allow a proximal control portion to expand from a distal anchor portion.

FIG. 9B is a close-up view of the slide components of FIG. 9A.

FIG. 10A is a cross-sectional view of an adjustable controller of the present disclosure including pull wire tensioners of the present disclosure that accommodate variable distances between portions of a handle of the controller.

FIG. 10B is a cross-sectional view of the adjustable controller of FIG. 10A with the handle in an extended state and the pull wire tensioners straightened out.

FIG. 11 is cross-sectional view of the adjustable controller of FIGS. 10A and 10B showing an irregular cross-sectional shape to prevent rotation.

FIG. 12 is a cross-sectional view of a controller of the present disclosure having a handle with misaligned end portions between which pull wire tensioners of the present disclosure extend.

FIG. 13 is a schematic perspective view of a controller of the present disclosure having a handle with disconnected end portions between which pull wire tensioners of the present disclosure extend.

### DETAILED DESCRIPTION

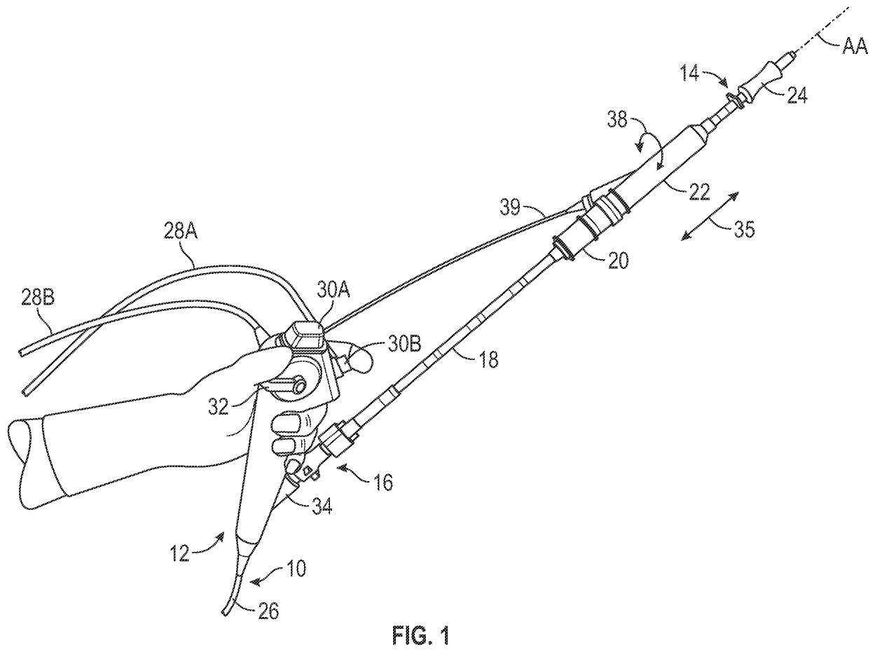

FIG. 1 is a schematic view of scope 10 having controller 12 to which can be connected catheter assembly 14. Catheter assembly 14 can comprise coupler 16, extension piece 18, rotation member 20, handle 22, and proximal grip 24. Scope 10 can comprise controller 12, scope shaft 26, input cable 28A, input cable 28B, button 30A, button 30B, lever 32 and working channel receptacle 34.

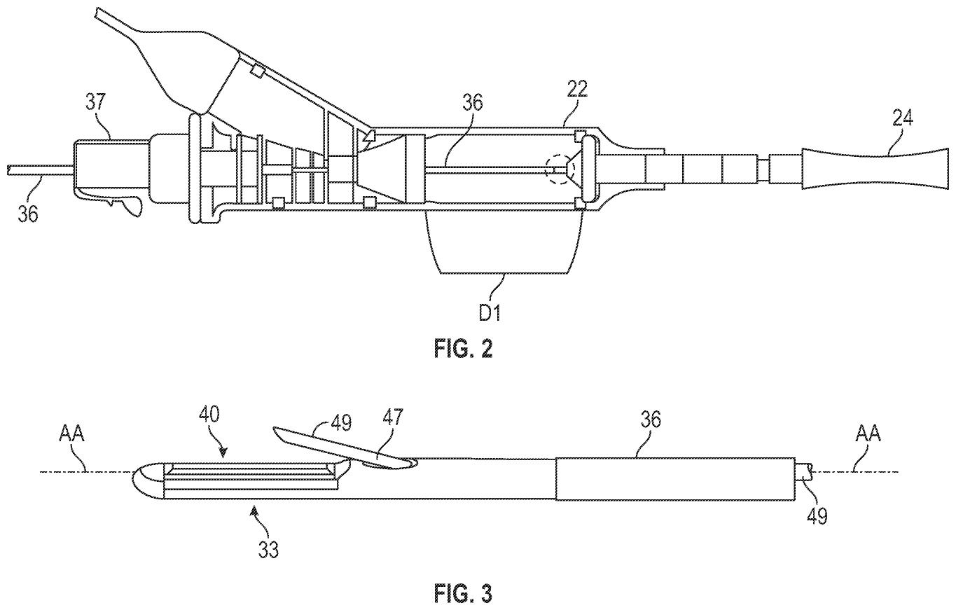

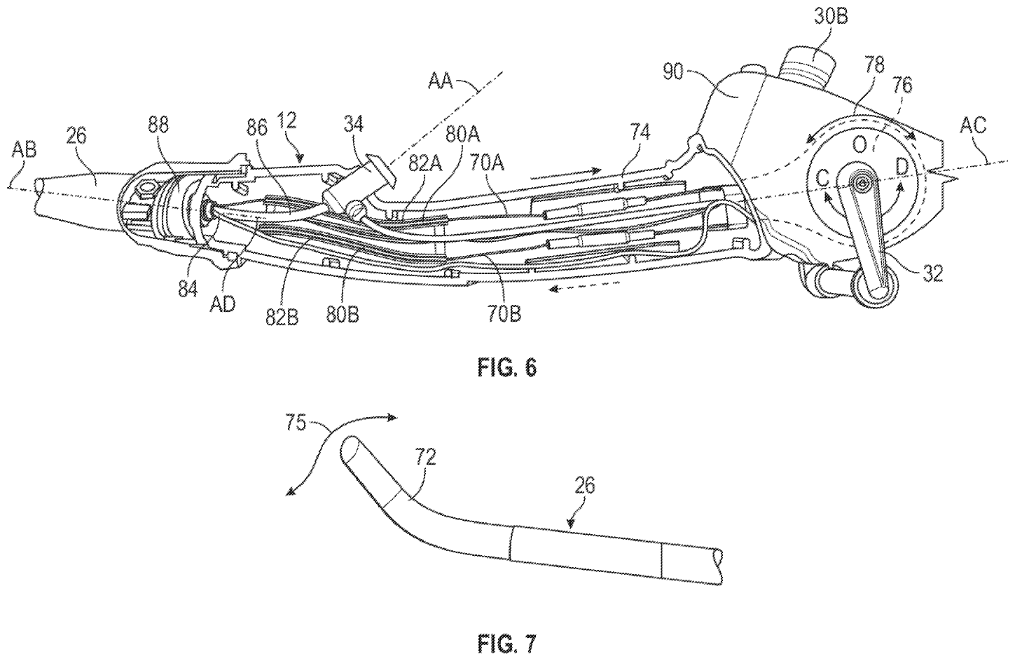

Catheter assembly 14, which is shown in more detail in FIG. 2 and FIG. 3, can comprise elongate catheter body 36 (FIG. 2) terminating in distal end section 33 (FIG. 3). Elongate catheter body 36 can extend from proximal grip 24, through rotation member 20, handle 22 and extension piece 18 and into coupler 16. Coupler 16 can be connected to working channel receptacle 34 of scope 10. Thus, elongate catheter body 36 can be inserted into working channel 84 (FIG. 6) of scope shaft 26 of scope 10. When assembled, elongate catheter body 36 and catheter assembly 14 can extend along central longitudinal axis AA.

Catheter assembly 14 can be manipulated independently of scope 10. Catheter assembly 14 can be manipulated with handle 22 and by rotation member 20. Handle 22 can be moved axially to move elongate catheter body 36 axially relative to scope 10 as indicated by arrow 35. Rotation member 20 can be rotated to rotate elongate catheter body 36 relative to scope 10 as indicated by arrow 38 can be accomplished via manipulation of rotation member 20 while scope 10 remains stationary.

Scope 10 can be used to perform an endobronchial intervention on a patient. For example, scope shaft 26 can be inserted perorally into a patient to extend into a lung. As discussed in greater detail below, scope shaft 26 can be guided to locations within the lungs via electromagnetic navigation. Handle 22 and rotation member 20 can be manipulated to navigate catheter assembly 14 and elongate catheter body 36 in particular, into a lung of a patient via scope 10. Thus, a physician can maneuver elongate catheter body 36 via scope 10 and proximal grip 24 and handle 22 of catheter assembly 14 to reach the desired anatomy. Lever 32 can be pushed and pulled to move the distal end of scope shaft 26, as shown in FIG. 7. Buttons 30A and 30B can be actuated to operate functions of scope 10. For example, one of buttons 30A and 30B can be actuated to operate imaging capabilities of scope 10 and the other of buttons 30A and 30B can be actuated to provide irrigation to scope shaft 26. In examples, input cable 28A can comprise a tube connected to a fluid source and input cable 28B can comprise an electrical cable connected to an electronics tower including imaging, video and treatment capabilities. Line 39 can connect input cable 28A to catheter assembly 14.

FIG. 2 is a schematic cross-sectional view of handle 22 for catheter assembly 14 of FIG. 1 showing elongate catheter body 36 extending therethrough.

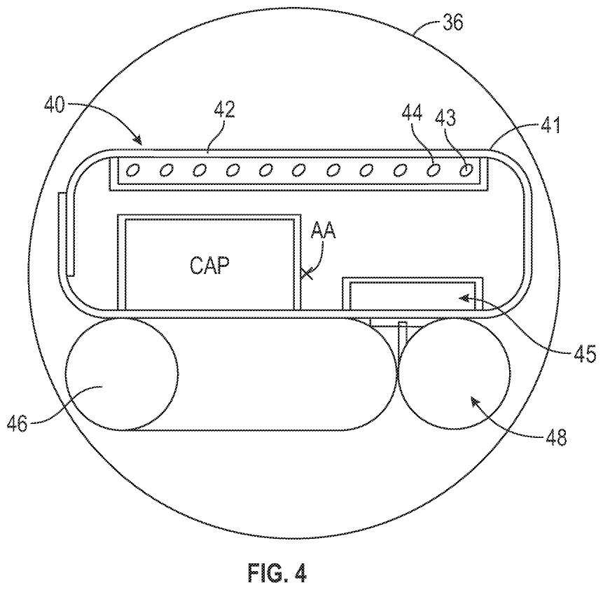

Extension piece 18 (FIG. 1) can be connected to hub 37. Extension piece 18 can extend between coupler 16 (FIG. 1) and rotation member 20 (FIG. 1). The length of extension piece 18 can be adjustable, e.g., via telescoping action, to allow handle 22 to be brought closer to scope 10. As such, portions of elongate catheter body 36 within extension piece 18 can be pushed further into scope 10 and anatomy. Handle 22 can be pushed and pulled relative to coupler 16 to provide macro adjustments to the position of elongate catheter body 36 along axis AA. Proximal grip 24 can be moved relative to handle 22 to provide micro adjustment to the position of elongate catheter body 36 along axis AA. In examples, proximal grip 24 can provide sufficient movement of elongate catheter body 36 to allow for extension and retraction of distal end section 33 (FIG. 3) into the distal end of scope shaft 26 of scope 10 and to effect small motions to sensors, such as piezoelectric transducers or elements 43 and 44 (FIG. 4). In examples, proximal grip 24 can move distance D1 within handle 22.

FIG. 3 shows distal end section 33 of elongate catheter body 36 in isolation from catheter assembly 14. Distal end section 33 can comprise functional components 40 and aperture 47 for tool 49. In examples, tool 49 can comprise a needle. Elongate catheter body 36 can include as lumen having a proximal access portal (not illustrated) and that can connect to aperture 47. The lumen can be used to introduce tool 49 into elongate catheter body 36 and scope 10.

FIG. 4 is a cross-sectional view of distal end section 33 of elongate catheter body 36. Distal end section 33 can include functional components 40. In examples, functional components 40 can comprise multi-element planar ultrasound array 41. Multi-element planar ultrasound array 41 can be positioned parallel to axis AA of catheter assembly 14 and can be covered by acoustic lens 42. Multi-element planar ultrasound array 41 can include a plurality of piezoelectric transducers or elements, such as element 43 and element 44, which can be fabricated into an array using micromachining technology. Electronics package 45 can be located in distal end section 33 near electromagnetic coil 46 and electromagnetic coil 48.

Portions of elongate catheter body 36 can be fabricated from stainless steel or other material. Elongate catheter body 36 can include a laser cut spiral pattern to facilitate flexibility in one or more planes.

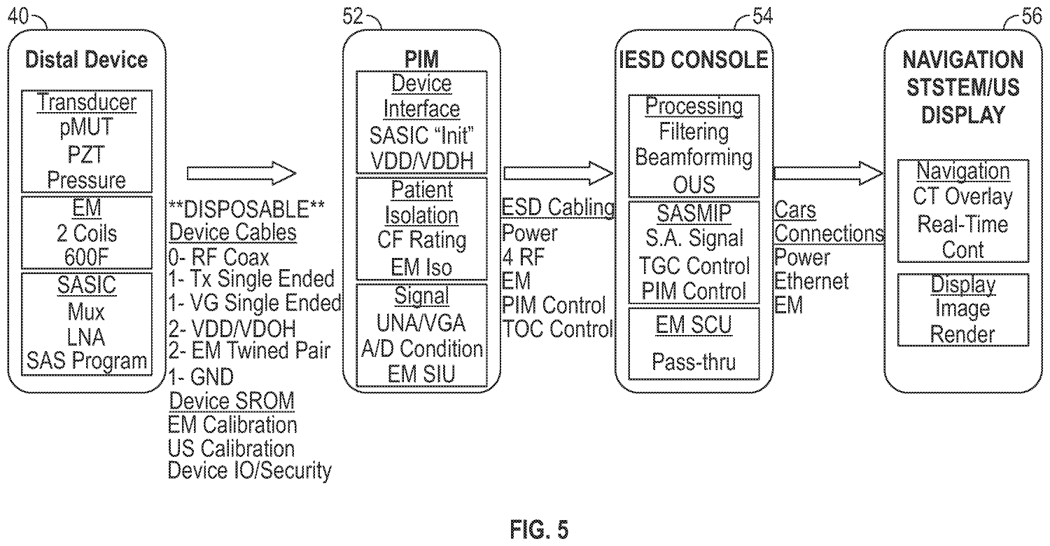

FIG. 5 is a partitioning of the electronic componentry of catheter assembly 14 and its accompanying imaging system and is an illustrative but not limiting version of the system in its present implementation.

The electronics package 45 of FIG. 4 can contain among other things a programmable chip to configure multi-element planar ultrasound array 41. A multiplexer can format and transmit data from catheter assembly 14 to the patient interface module 52 (or PIM 52), which can be hung bedside on a gurney with the patient. PIM 52 can include electrical isolation to protect the patient and also contain power supplies for catheter assembly 14 itself, A/D conversion and various buffering processes can be accomplished in PIM 52 to improve noise performance of catheter assembly 14. In this implementation a separate “pizza box” enclosure can carry dedicated hardware for the synthetic aperture beam forming and control as well as the spectral analysis of the backscattered signals for the QUS processes 54. The enclosure is coupled to the workstation-based navigation and display cart. The QUS backscatter evaluation system residing in the pizza box enclosure can be separate or incorporated into workstation 56 itself where visual images of the ultrasonic signals and analysis is displayed.

Elongate catheter body 36 can carry a matrix of individually addressable piezoelectric transducers or elements fabricated into multi-element planar ultrasound array 41 using micromachining technology. Each element of multi-element planar ultrasound array 41, illustrated in FIG. 4 as elements 43 and 44, can be powered to emit ultrasonic energy as a spherical wave emanating from the specific transducer location, and each element in the array can function as a receiver transducing the mechanical energy of backscattered sound into an electrical signal. Once a spherical wave is launched from a given element 43, a companion transducer, such as element 44, can detect after a delay, the backscattered energy reflected off of biologic tissues. In the synthetic aperture scenario only one element 44 is listening to element 43 element at a time.

In general pairs of elements will be activated with one element 43 functioning as a transmitter of acoustic energy and another element 44 functioning as a receiver. Since the elements are arrayed in space several viewpoints are present in the array. This provides much improved lateral resolution when compared to prior art approaches.

With that data stored, a next transducer in the array is activated to transmit acoustic energy and its complimentary transducer receives the backscattered return signal. With many, for example sixty-four transducers, at various locations, the composite of all the returned energy from all the locations can be used to form though computation an image plane orthogonal to the plane of the transducer.

It is possible to have more than one transducer pair active at a time and in the exemplary embodiment four channels of data can be collected synchronously. The limitations are based on complexity and power dissipation and bandwidth of the data paths. Consequently, other configurations are possible and anticipated within the scope of the claims. The mathematics to pull an image in a plane from the time sequenced multiplex data that is transmitted and received at various points in space is complicated but well known and understood in the field. In general, the displayed image plane is synthesized from data taken at many locations in space taken at different times, that are collectively convolved into a single image plane hence the term synthetic aperture. If one moves the catheter along a path the synthetic aperture image plane sweeps out a volume. This is a relatively low-resolution image of a volume of tissue but can help to resolve the extent of anatomy to supplement the detection of anatomic structures such as airways, blood vessels, and the like in the 2-D first image plane area. In this regard the methodology of the invention may rely on a first target image area in a plane or rely on a 3-D volume called the first target image volume. In this later case catheter movement is used to define the first 3-D volume of target tissue.

In use there are two modes of operation for the ultrasound transducer array. In a first mode, the amplitude and envelope information from the backscattered acoustic energy is used to form an image presented to the clinician. This may be a first 2-D slice of target tissue or a 3-D volume of target tissue. In a second mode the transmitted power is reduced to select a smaller target plane or volume within the first image plane or image volume. This reduced view is called the second reduced area or slice in the event of a 2-D slice or a second reduced volume in the event of a 3-D volume. In each case the reduced view is selected to be free of anatomic detail observed in the first view. The exclusion of gross anatomic structure selects a homogenous sample for quantitative analysis. The spectra of the backscattered energy from the reduced area slice or volume is evaluated quantitatively and automatically rather than used to form an image. The image free quantitative information is used to determine if the reduced area of tissue exhibits the acoustic characteristics of cancerous tissue. The precise characteristics or the acoustics of cancer is a topic of study at the present time.

FIG. 6 is side view of controller 12 of scope 10 of FIG. 1 showing pull wire 70A and pull wire 70B for scope shaft 26 of scope 10. FIG. 7 is a perspective view of distal tip 72 of scope shaft 26 of scope 10 of FIG. 6. FIGS. 6 and 7 are discussed concurrently.

As discussed, catheter assembly 14 (FIG. 1) can be connected to working channel receptacle 34 and can extend along axis AA. Scope shaft 26 can extend from housing 74 of controller 12 along central longitudinal axis AB. As such, axis AA and axis AB can be concentric along portions of elongate catheter body 36 extended into scope shaft 26.

Distal tip 72 can be rotated about axis AB by rotation of controller 12. Distal tip 72 can be articulated, e.g., bent along axis AB, in a plane indicated by arrow 75 (FIG. 7). In particular, pull wire 70A and pull wire 70B can be connected to lever 32 (FIG. 1) via gear 76 shown in phantom. Thus, movement of lever 32 can rotate gear 76 in the direction of arrow 78, thereby causing movement of distal tip 72 in the direction of arrow 75. In examples, movement of lever 32 to the left in FIG. 6 can cause pull wire 70A to pull on distal tip 72 causing upward movement of distal tip 72 in FIG. 7, and movement of lever 32 to the right in FIG. 6 can cause pull wire 70B to pull on distal tip 72 causing downward movement of distal tip 72 in FIG. 7. In order to facilitate movement of pull wires 70A and 70B within controller 12, pull wires 70A and 70B can be disposed within sheaths 80A and 80B, respectively. Sheath 80A and sheath 80B can be fixedly mounted to housing 74 of controller 12. Sheaths 80A and 80B can help maintain pull wires 70A and 70B aligned with working channel 84 of scope shaft 26. Sheaths 80A and 80B can additionally apply compression to pull wires 70A and 70B to help maintain distal tip 72 in the orientation applied by lever 32. Sheaths 80A and 80B can be coupled to housing 74 via support 82A and support 82B, respectively. Supports 82A and 82B can support the substantial majority of sheath 80A and sheath 80B, particularly along the central portions of sheaths 80A and 80B. Support 82A and support 82B can immobilize sheath 80A and sheath 80B within housing 74 and can prevent bending of sheaths 80A and 80B.

Working channel 84 of scope shaft 26 can be connected to working channel receptacle 34 by tube 86. In particular, scope shaft 26 can extend into anchor portion 88 so that working channel 84 of scope shaft 26 is within housing 74. Thus, tube 86 can connect working channel 84 with working channel receptacle 34. Working channel receptacle 34 can be connected to coupler 16 of catheter assembly 14, as shown in FIG. 1. Working channel receptacle 34 can be located in close proximity to anchor portion 88. Lever 32 and button 30B can be located at an opposite end of housing 74 than anchor portion 88 at control portion 90.

For the configuration of FIG. 6, controller 12 can be arranged so that proximal portions of pull wire 70A and pull wire 70B extend along axis AC from gear 76 and control portion 90. Distal portions of pull wire 70A and pull wire 70B can extend along axis AD, which can be coincident with axis AB or scope shaft 26 at anchor portion 88. Axis AC can be disposed at an angle of approximately one-hundred-eighty-degrees to approximately one-hundred-fifty degrees. As such, working channel 84 can be axially aligned with gear 76 or have a line-of-sight with gear 76. Thus, sheaths 80A and 80B are fixed in positions to guide pull wires 70A and 70B from gear 76 to working channel 84. It is impractical to include kinks, e.g., bends greater than approximately thirty degrees, within sheaths 80A and 80B because pull wires 70A and 70B can become stressed therein when subject to pulling. Furthermore, it is not possible to adjust the position between lever 32 and working channel 84 because pull wires 70A and 70B would become overstretched or have slack introduced therein.

As shown in FIG. 1, handle 22 for catheter assembly 14 can be located a variable distance from working channel receptacle 34 depending on the state of extension piece 18. Regardless of the length of extension piece 18, handle 22 can be located a distance away from lever 32 above or proximal to lever 32 and laterally offset from lever 32. Further, lever 32 can be located a fixed distance form working channel receptacle 34. As such, the operation of scope 10 and catheter assembly 14 can be cumbersome due to positioning of controller 12 away from handle 22.

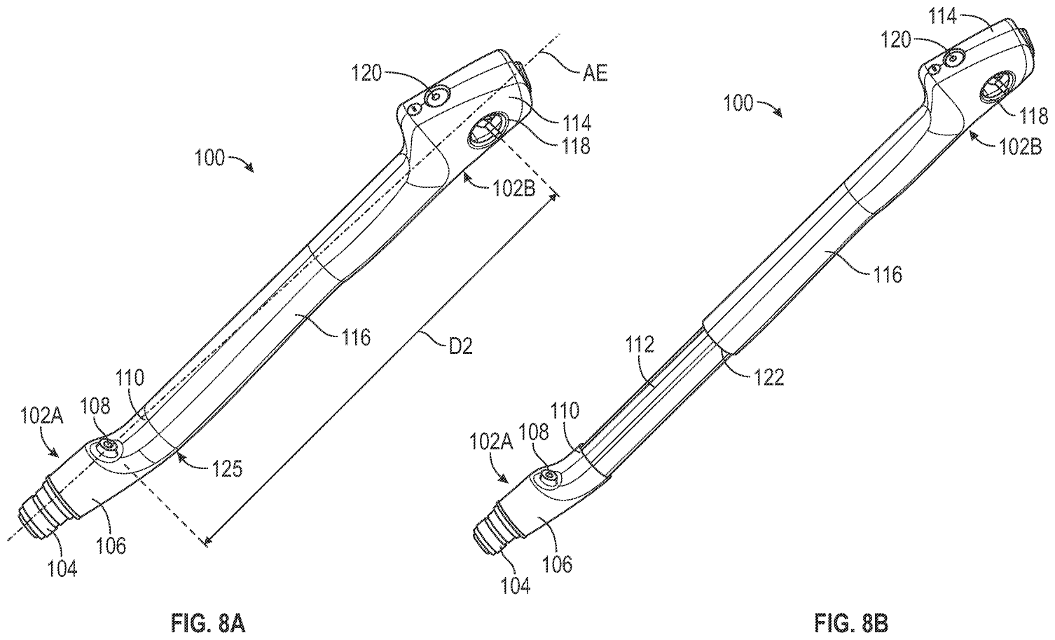

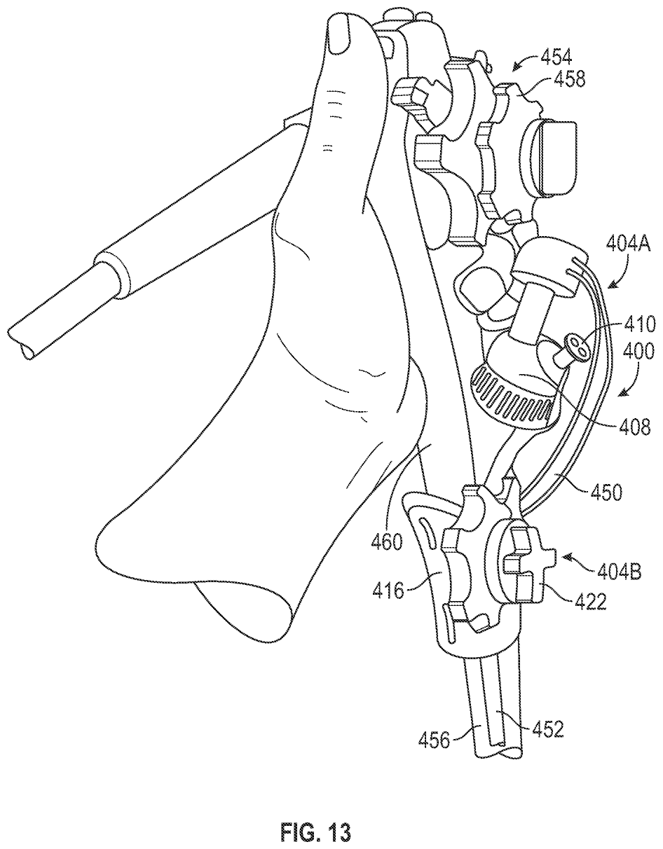

With the present disclosure, controllers for scopes, such as controller 12, can be configured to have 1) actuation mechanisms, e.g., buttons and levers, that are adjustably positionable relative to each other, 2) controller housings that have portions that are adjustably positionable relative to each other [FIGS. 8A-10B], 3) controller housings that can have anchor portions and control portions that are bent, curved, kinked or otherwise configured such that a gear for pull wires is not aligned or does not have line-of-sight with a working channel opening of a scope shaft [FIG. 12], and 4) controller housings that can be split apart into two different portions located away from each other [FIG. 13] or have a flexible handle portion disposed therebetween. In order to facilitate such configurations, controller housing of the present disclosure can include pull wire tensioners that allow pull wires to be bent, curved, kinked, pulled, pushed or otherwise include irregular or changing paths to connect misaligned, disconnected or adjustable portions of a controller housing, while maintaining tension in the pull wires to eliminate slack and provide other advantages as described herein.

FIG. 8A is perspective view of adjustable controller 100 of the present disclosure in a retracted state. FIG. 8B is a perspective view of adjustable controller 100 of FIG. 8A in an extended state. FIGS. 8A and 8B are discussed concurrently.

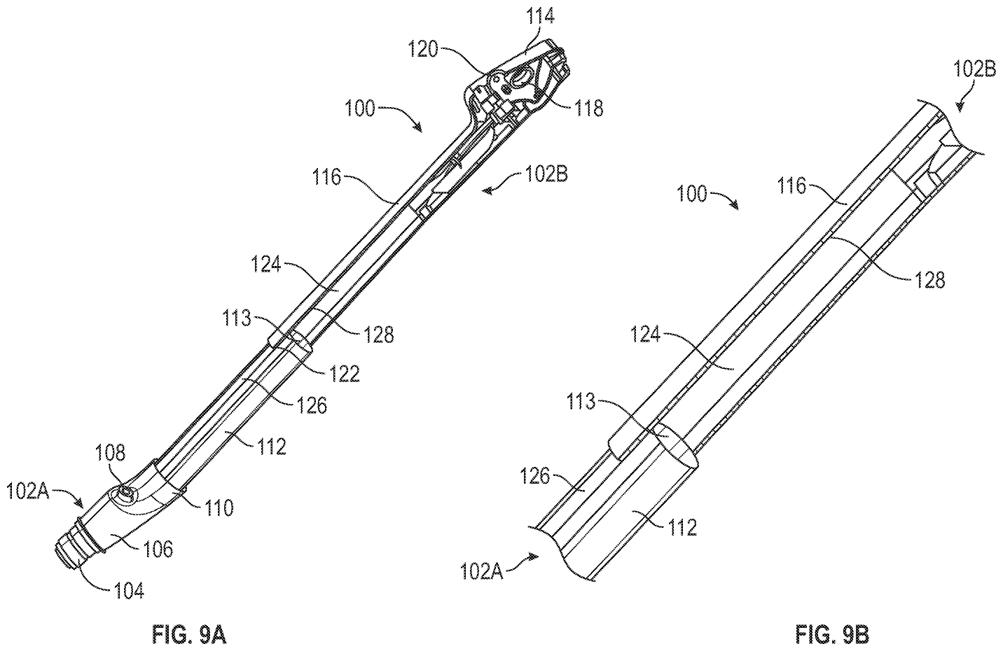

Adjustable controller 100 can comprise anchor component 102A and controller component 102B. Anchor component 102A can comprise fastener 104, base 106, working channel aperture 108, stop 110, slide post 112 and internal passage 113 (FIGS. 9A and 9B). Controller component 102B can comprise head 114, slider component 116, lever aperture 118, button aperture 120, end 122 and internal passage 124 (FIGS. 9A and 9B).

Controller component 102B can be configured to move relative to anchor component 102A. In the illustrated example, controller component 102B can slide axially relative to anchor component 102A along axis AE. As such, distance D2 between working channel aperture 108 and lever aperture 118 can be adjusted. For example, a user can move controller component 102B away from anchor component 102A to increase distance D2 so that head 114 is located in a comfortable position for use.

Fastener 104 can be coupled to an elongate flexible shaft configured to be inserted into anatomy of a patient. Fastener 104 can comprise a threaded engagement or a lip, flange, channel or another feature to which a component of a scope shaft can mate. Base 106 can comprise a structural component that can support other portions of adjustable controller 100 including working channel aperture 108 and controller component 102B. Similar to what is shown in FIG. 1, working channel aperture 108 and base 106 can be configured to support another instrument in a fixed or working relationship relative to adjustable controller 100. Slide post 112 can extend from base 106 at stop 110. Slide post 112 can comprise an elongate portion of anchor component 102A upon which controller component 102B can slide. In the illustrated example, slide post 112 can comprise a reduced-diameter portion relative to base 106. Stop 110 can be configured to abut controller component 102B in a fully collapsed position when D2 is smallest, as shown in FIG. 8A.

Head 114 can comprise a structural component for receive control actuators for adjustable controller 100. For example, lever aperture 118 can receive a pull wire lever similar to lever 32 (FIG. 1) and button aperture 120 can receive a button similar to button 30B (FIG. 1) to control functions of a scope, such as imaging capabilities or irrigation capabilities and the like. Head 114 can be shaped to allow a user to grasp controller component 102B to operate the lever and button. Slider component 116 can extend from head 114 to engage with anchor component 102A. Slider component 116 can comprise an elongate tube configured to receive slide post 112. In the illustrated example, slider component 116 can comprise a reduced-diameter portion relative to head 114. End 122 of slider component 116 can be configured to engage stop 110 of anchor component 102A at interface 125. Slider component 116 can be configured to provide one or more locations for a user to place fingers for a hand that operates actuators at lever aperture 118 and button aperture 120 as well as another hand.

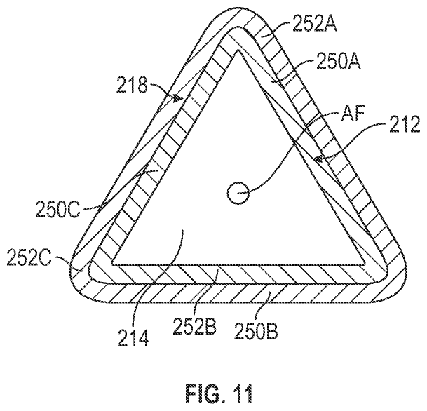

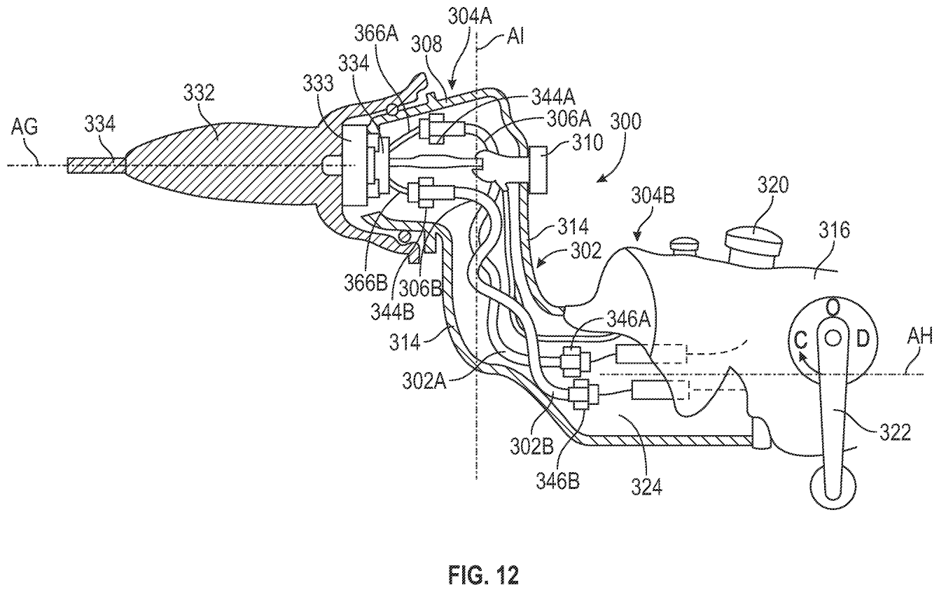

Slide post 112 and slider component 116 can comprise hollow tubular bodies through which pull wires can extend. For example, pull wires can extend from a gear connected to a lever at lever aperture 118 to a scope shaft connected to fastener 104. Slide post 112 can have an outer perimeter shape that is complementary to an inner perimeter shape of slider component 116. In the illustrated example of FIGS. 8A and 8B, slide post 112 and slider component 116 can have circular cross-sectional shapes. However, in other examples, other cross-sectional shapes can be used, as shown and discussed with reference to FIG. 11.

FIG. 9A is a partially broken-away perspective view of adjustable controller 100 of FIG. 8B showing slide post 112 and slider component 116 that are configured to allow controller component 102B to expand away from anchor component 102A. FIG. 9B is a close-up view of slide post 112 and slider component 116 of FIG. 9A. Slide post 112 can be fitted into internal passage 124. Slide post 112 can comprise channel 126 and internal passage 124 can comprise flange 128 that can reside within channel 126. FIGS. 9A and 9B are discussed concurrently.

As discussed, slide post 112 and slider component 116 can be configured to have circular cross-sectional profiles. As such, relative rotation between slide post 112 and slider component 116 along axis AE (FIG. 8A) can be permitted. However, in order to hold the relative rotational positions of slide post 112 and slider component 116, anti-rotation features can be included. In example, channel 126 can extend parallel to axis AE along at least a portion of the length of slide post 112. Likewise, flange 128 can extend parallel to axis AE along at least a portion of the length of slider component 116. Flange 128 can ride within channel 126 when anchor component 102A and controller component 102B are assembled. Flange 128 can permit controller component 102B to move axially relative to anchor component 102A. However, flange 128 can prevent or inhibit, or at least limit, the amount of relative rotation that controller component 102B can undergo relative to anchor component 102A. In examples, flange 128 can be almost as wide in the circumferential direction relative to axis AE as channel 126 so that circumferential rotation is prevented, while still allowing axial movement. In other examples, channel 126 can be provided on slider component 116 and flange 128 can be included on slide post 112.

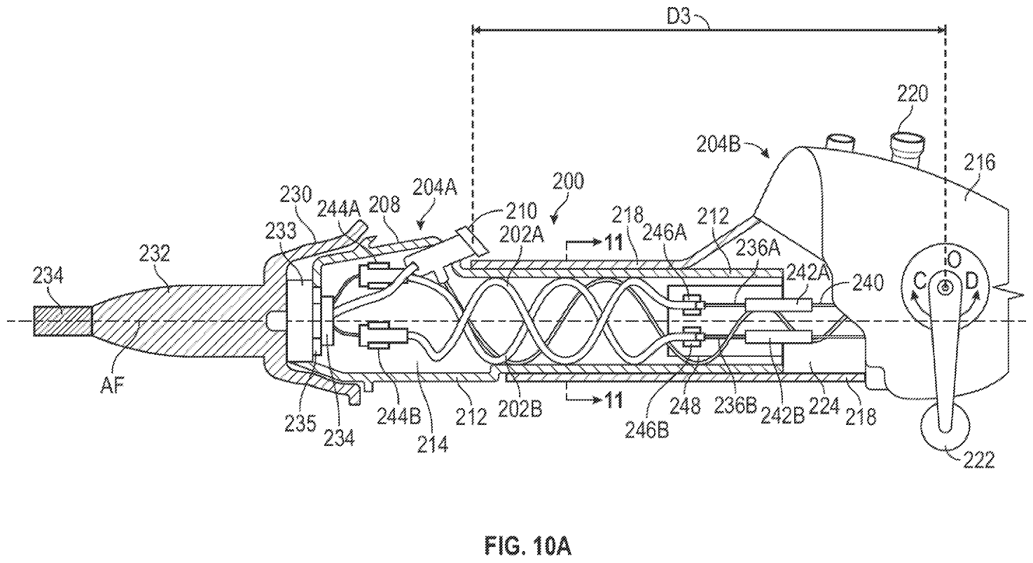

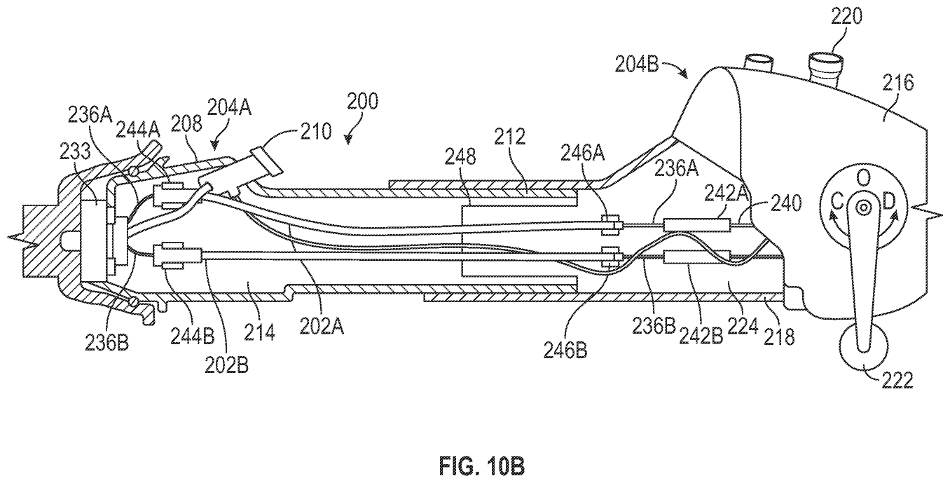

FIG. 10A is a cross-sectional view of adjustable controller 200 of the present disclosure including pull wire tensioner 202A and pull wire tensioner 202B of the present disclosure that accommodate variable distances between anchor component 204A and controller component 204B of adjustable controller 200. FIG. 10B is a cross-sectional view of adjustable controller 200 of FIG. 10A with controller component 204B extended from anchor component 204A. FIGS. 10A and 10B are discussed concurrently.

Anchor component 204A can comprise base 208, working channel aperture 210, slide post 212 and internal passage 214. Controller component 204B can comprise head 216, slider portion 218, button 220, lever 222 and internal passage 224. In examples, adjustable controller 200 can be configured similarly to controller 12 of FIG. 6 with housing 74 split into anchor component 204A and controller component 204B. In examples, anchor component 204A and controller component 204B can be configured similarly as anchor component 102A and controller component 102B of FIGS. 8A-9B.

Cap 230 can be connected to base 208 to attach scope shaft 234 to anchor component 204A. Strain relief 232 can be connected to cap 230 via flange 233. Strain relief can comprise a flexible tube for limiting flexure of scope shaft 234 and thereby reducing strain generated therein. In examples, strain relief 232 can extend through an opening in cap 230 and flange 233 can prevent strain relief from passing through cap 230. Cap 230 can be threadedly attached to base 208. Scope shaft 234 can pass into flange 233 and through strain relief 232. Scope shaft 234 can be crimped to an opening in base 208 using swage 235. Scope shaft 234 can, however, be connected to anchor component 204A in a variety of ways.

Scope shaft 234 can extend from swage 235, through cap 230 and strain relief 232 to a distal end portion, similar to distal tip 72 of scope shaft 26 shown in FIG. 7. Pull wires 236A and 236B can extend to the distal portion of scope shaft 234 to induce bending or curvature of the distal tip portion. Pull wire 236A and pull wire 236B can extend from scope shaft 234 within internal passage 214 of anchor component 204A. Pull wires 236A and 236B can continue to a gear (now shown; similar to gear 76 of FIG. 6) within head 216 that is connected to lever 222. Note, in examples, pull wire 236A and 236B can be connected to wire segment 240 via connectors 242A and 242B, respectively, for manufacturing purposes. As such, lever 222 can be actuated to pull on each of pull wires 236A and 236B. With reference to FIG. 7, in examples, movement of lever 32 to the left in FIG. 10A can cause pull wire 236A to pull on distal tip 72 causing upward movement of distal tip 72 in FIG. 7, and movement of lever 32 to the right in FIG. 10A can cause pull wire 236B to pull on distal tip 72 causing downward movement of distal tip 72 in FIG. 7.

Pull wire 236A can extend into wire tensioner 202A and pull wire 236B can extend into wire tensioner 202B. Wire tensioner 202A can be connected to slide post 212 via anchor 244A and wire tensioner 202B can be connected to slide post 212 via anchor 244B. Wire tensioner 202A can be connected to slider portion 218 via anchor 246A and wire tensioner 202B can be connected to slider portion 218 via anchor 246B. Each of wire tensioner 202A and wire tensioner 202B can comprise a component or device for tacking up slack and inducing tension within pull wires 236A and 236B, respectively. Wire tensioner 202A and wire tensioner 202B can be used to accommodate a change in distance between portions or components of adjustable controller 200. In the illustrated example, adjustable controller 200 can include anchor component 204A and controller component 204B that can be translated away from anchor component 204A along axis AF. As such, head 216 and lever 222 can be moved away from base 208 and working channel aperture 210. However, in other examples, lever 222 can be configured to move relative to working channel aperture 210 without controller component 204B being moveable relative to anchor component 204A. Thus, in the various configurations, lever 222 can be positioned distance D3 away from working channel aperture 210. In FIG. 10B, distance D3 can be increased over distance D3 in FIG. 10A by translating, e.g., sliding, controller component 204B away from anchor component 204A.

As discussed herein, in conventional scope controllers having pull wires, it is not possible to move a pull wire actuator away from the location where the pull wires enter into a scope shaft because there is not slack within the pull wires and slack cannot be introduced without diminishing the capabilities of the pull wire actuator. With the present disclosure, adjustable controller 200 can include pull wire tensioner 202A and pull wire tensioner 202B to allow tension to be maintained within pull wire 236A and pull wire 236B, respectively, which allows slack, e.g., length in excess of what is needed to connect two points by the shortest route, to be introduced into pull wires 236A and 236B to facilitate movement of lever 222 away from base 208. The tension applied by pull wire tensioners 202A and 202B can eliminate play or slop in movement of lever 222 before actual pulling of pull wires 236A and 236B begins due to the slack. In other words, pull wire tensioners 202A and 202B can allow lever 222 to provide immediate responsiveness in applying tension to pull wires 236A and 236B even though there is excess length of pull wires 236A and 236B.

A first end of pull wire tensioner 202A can be connected to anchor component 204A at anchor 244A and a second end of pull wire tensioner 202A can be connected to controller component 204B at anchor 246A. A first end of pull wire tensioner 202B can be connected to anchor component 204A at anchor 244B and a second end of pull wire tensioner 202B can be connected to controller component 204B at anchor 246B. Anchor component 204A can include a cut-out or window 248 to allow anchors 246A and 246B to move closer to anchors 244A and 244B within anchor component 204A. Anchors 244A, 244B, 246A and 246B can comprise clips or U-shaped bodies into which pull wire tensioners 202A and 202B can be fitted. Pull wire tensioners 202A and 202B can be adhered or glued to anchors 244A, 244B, 246A and 246B to immobilize ends of pull wire tensioners 202A and 202B relative to their respective handle components. However, pull wire tensioners 202A and 202B can be unsupported or free-floating between anchors 244A and 244B and anchors 246A and 246B to allow for expansion, straightening out or flattening of curves within pull wire tensioners 202A and 202B.

Pull wire tensioner 202A and pull wire tensioner 202B can comprise ferrules or curved elongate tubes through which pull wire 236A and pull wire 236B can extend, respectively. The elongate tubes can be sufficiently rigid such that pulling of pull wires 236A and 236B does not deform, e.g., un-curve or straighten, the elongate tubes, but the elongate tubes can be sufficiently flexible such that pulling of controller component 204B away from anchor component 204A can bend the elongate tubes into more straight or less curved bodies. In examples, pull wire tensioners 202A and 202B can be fabricated from plastic or metal. In examples, pull wire tensioners 202A and 202B can each include one long curve between anchors. In examples, pull wire tensioners 202A and 202B can be undulating, e.g., have multiple up and down curves, between anchors. As such, the absolute distance between a first end of pull wire tensioner 202A at anchor 244A and a second end of pull wire tensioner 202A at anchor 246A can be increased as the undulations become muted or less curved. In other words, the lengths along a central axes of pull wire tensioner 202A and pull wire tensioner 202B can be longer than the distances between anchors 244A and 244B and 246A and 246B, respectively, in the retracted state, but the lengths along the central axis of pull wire tensioners 202A and 202B can be increased to be close to or equal to the distance between anchors 244A and 244B and 246A and 246B in the extended state. The inherent rigidity of pull wire tensioner 202A and pull wire tensioner 202B can maintain tension on pull wires 236A and 236B to maintain responsiveness of lever 222. The curved or undulating nature of pull wire tensioner 202A and 202B can allow for the length of adjustable controller to be varied or increased or the distance between working channel aperture 210 and lever 222 to be varied or increased. However, pull wire tensioners 202A and 202B can additionally be used to offset or uncouple controller component 204B from anchor component 204A, as discussed with reference to FIGS. 12 and 13, respectively.

FIG. 11 is cross-sectional view of adjustable controller 200 of FIG. 10A showing an irregular cross-sectional shape to prevent rotation. Slide post 212 of anchor component 204A can be situated within slider portion 218 of controller component 204B. In the example of FIG. 11, slide post 212 and slider portion 218 can have hollow triangular cross-sectional profiles. For example, slide post 212 can comprise first panel 250A, second panel 250B and third panel 250C and slider portion 218 can comprise first panel 252A, second panel 252B and third panel 252C. First panel 250A, second panel 250B and third panel 250C of slide post 212 can fit within internal passage 224 slider portion 218 and internal passage 214 of slide post 212 can be open to accommodate pull wires 236A and 236B and pull wire tensioners 202A and 202B, which are not shown in FIG. 11 for simplicity. The irregular cross-sectional profiles of slide post 212 and slider portion 218 can prevent relative rotation between slide post 212 and slider portion 218, thereby allowing adjustable controller to maintain relative rotational positioning of various features, such as working channel aperture 210 and lever 222.

In additional examples of the present disclosure, slide post 212 and slider portion 218 can have a regular, e.g., circular, cross-sectional profiles to allow for relative rotation therebetween. In such example, adjustable controller 200 can include a selectively actuated lock feature to allow the rotational positions to be immobilized at a desired location by a user. For example, slide post 212 can include a spring loaded detent and slider portion 218 can include a plurality of holes at different circumferential positions relative to central axis AF.

FIG. 12 is a cross-sectional view of controller 300 of the present disclosure having handle 302 with misaligned anchor portions 304A and controller portion 304B between which pull wire tensioners 306A and 306B of the present disclosure extend. Controller 300 can be configured similarly as controller 200 of FIGS. 10A and 10B with anchor portion 304A being connected to controller portion 304B by fixed middle portion 314 instead of anchor component 204A being slidably connected to controller component 204B. As such, controller 300 is provided with similar reference numbers as controller 200, but with different series numbers, e.g., 300 numbers rather than 200 numbers.

Controller 300 can comprise base 308, working channel aperture 310, middle portion 314, head 316, button 320, lever 322, internal passage 324, strain relief 332 and flange 333. Controller 300 can further comprise pull wires 336A and 336B, which can be inserted in pull wire tensioners 302A and 302B, respectively. Pull wire tensioners 302A and 302B can be connected to anchors 344A and 344B and 346A and 346B, respectively.

Handle 302 can comprise anchor portion 304A and controller portion 304B, which can be connected by middle portion 314. Anchor portion 304A can extend along axis AG, controller portion 304B can extend along axis AH and middle portion 314 can extend along axis AI. In the illustrated example, axis AG can extend parallel to axis AH, with axis AI being perpendicular to axes AG and AH. However, in other examples, axes AG and AH can be oblique to each other, and axes AG and AH can be oblique to axis AI. In examples, the angles between axes AG, AH and AI can be such that the opening of scope shaft 334 and working channel aperture 310 do not have line-of-sight with the gear for lever 322 within controller 300, e.g., through internal passage 324. For example, axis AG and axis AI can be disposed at an angle greater than approximately thirty degrees and axis AI and axis AH can be disposed at an angle greater than approximately thirty degrees. Pull wire tensioners 306A and 306B can be used to thread pull wires 336A and 336B through middle portion 314 to maintain tension therein to allow lever 322 to responsively pull on pull wires 336A and 336B, while additionally preventing binding of pull wires 336A and 336B against surfaces of base 308, middle portion 314 and head 316.

Anchors 344A and 344B and 346A and 346B can hold ends of pull wire tensioners 302A and 302B fixed while pull wire tensioners 302A and 302B can remain floating therebetween. Ends of pull wire tensioners 302A and 302B can be oriented to face the working channel for scope shaft 334 and lever 322 to facilitate smooth movement of pull wires 336A and 336B. In examples, pull wire tensioners 302A and 302B can follow the general path of axes AH, AI and AG, while simultaneously being undulating along such path. Pull wire tensioners 302A and 302B can take up excess length, or slack, within pull wires 336A and 336B to allow pull wires 336A and 336B to extend between scope shaft 334 and lever 332 without binding along portions, e.g., corners, of base 308, middle portion 314 and head 316.

FIG. 13 is a schematic perspective view of controller 400 of the present disclosure having anchor component 404A disconnected from controller component 404B. Pull wire tensioners of the present disclosure can extend between anchor component 404A and controller component 404B within tube 450.

Controller 400 can be configured similarly as controller 200 of FIGS. 10A and 10B with anchor component 404A being separated from controller component 404B instead of anchor component 204A being slidably connected to controller component 204B. Anchor component 404A can comprise base 408 and working channel aperture 410. Controller component 404B can comprise head 416 and knob 422. Anchor component 404A can be linked to controller component 404B by tube 450. Head 416 can include cable 452 for connecting to electronics, a surgical system tower, electrical generator, imaging equipment and the like.

Controller 400 can be connected to another medical device or scope, such as duodenoscope 454. Duodenoscope 454 can comprise shaft 456, which can be inserted into anatomy of a patient and can include a working channel into which a shaft for controller 400 can be inserted. Thus, a tool or instrument, such as a catheter, can be inserted into working channel aperture 410 to be inserted into the shaft for controller 400 located inside of shaft 456. Knob 458 of duodenoscope 454 can be operated to curve or bend shaft 456. Knob 422 can be operated to curve or bend the distal tip of the shaft of controller 400 that protrudes from shaft 456.

Base 408 can be coupled to handle 460 of duodenoscope 454. In examples, base 408 can comprise a cap that can be threadedly engaged with an access portion on handle 460 of duodenoscope 454. Thus, base 408 can provide a direct entry way into handle 460 for tube 450 and working channel aperture 410. However, head 416 can be located remotely from base 408 and can thereby be positioned in a more convenient or ergonomic position as opposed to merely extending from base 408. Head can comprise a hollow body for holding actuation components for controller 400. In examples, head 416 can be held by an operator. In the illustrated example, base 408 can be attached to handle 460. For example, base 408 can comprise a U-shaped clip to snap onto a distal portion of handle 460. Thus, pull wires can extend from a gear attached to knob 422 within head 416 to base 408 via tube 450. In order to facilitate separation of head 416 from base 408, tube 450 can be provided with pull wire tensioners of the present disclosure. Thus, tensioner anchors can be attached to head 416 and base 408 and a tensioner tube or ferrule as disclosed herein can extend therebetween within tube 450. The tensioner tubes or ferrules can be curved or undulated to take out slack within the pull wires. However, the curvature or undulation of the tensioner tubes or ferrules can bend to allow head 416 to be moved into different positions relative to base 408. Thus, controller 400 can be similar to controller 300, but with tube 450 replacing middle portion 314. In additional examples of the present disclosure shown in FIG. 12, middle portion 314 can be flexible to allow for variable positioning of head 316 relative to base 308.

In view of the foregoing disclosure, the present disclosure can provide systems, devices and methods relating to ergonomic controls for endoscopes and other scopes. In particular, the present disclosure provides scope controllers having adjustably positionable controller components or control actuators. As such, the position between two actuators or the position between actuators and a coupling feature for an auxiliary scope can be adjusted to provide more ergonomic and user-friendly positioning. Additionally, the present disclosure can provide systems, devices and methods relating to pull wires that can be tensioned without line-of-sight between the control actuators and the working channel. As such, the handpiece for the scope controller can separate the control actuators and the working channel into offset handpiece components, moveable handpiece components or separated handpiece components.

### EXAMPLES

Example 1 is a controller for an endoscope, the controller comprising: a handle; a working channel opening connected to the handle at a first location; a control actuator connected to the handle at a second location; a first pull wire extending from the control actuator to the working channel opening within the handle; and a first pull wire tensioner through which the first pull wire extends; wherein the first pull wire tensioner is configured to remove slack from the first pull wire between the first location and the second location.

In Example 2, the subject matter of Example 1 optionally includes wherein the control actuator is configured to pull the first pull wire to bend an insertion shaft extending from the handle, the insertion shaft including a working channel connected to the working channel opening.

In Example 3, the subject matter of Example 2 optionally includes wherein the first pull wire tensioner comprises a curved tube.

In Example 4, the subject matter of Example 3 optionally includes wherein the first pull wire tensioner is fabricated from a rigid material and is flexible at curvature of the curved tube.

In Example 5, the subject matter of any one or more of Examples 3-4 optionally include wherein the first pull wire tensioner comprises: a first anchor fixed to the handle proximate the working channel opening; and a second anchor fixed to the handle proximate the control actuator.

In Example 6, the subject matter of Example 5 optionally includes wherein the first pull wire tensioner is floating within the handle along a majority of a length of the first pull wire tensioner.

In Example 7, the subject matter of any one or more of Examples 5-6 optionally include wherein a length along a central axis of the first pull wire tensioner between the first anchor and the second anchor is longer than a straight-line distance between the first anchor and the second anchor.

In Example 8, the subject matter of any one or more of Examples 5-7 optionally include wherein the curved tube is undulating.

In Example 9, the subject matter of any one or more of Examples 5-8 optionally include wherein the handle includes a kink between the first anchor and the second anchor.

In Example 10, the subject matter of Example 9 optionally includes wherein the handle includes a bend between the first location and the second location such that the first pull wire is turned at least thirty degrees.

In Example 11, the subject matter of any one or more of Examples 5-10 optionally include wherein the handle comprises: an anchor component connected to the working channel opening and the first anchor; and a control component connected to the control actuator and the second anchor.

In Example 12, the subject matter of Example 11 optionally includes wherein the anchor component and the control component are unconnected to each other and the first pull wire tensioner extends therebetween.

In Example 13, the subject matter of Example 12 optionally includes wherein the anchor component and the control component are connected by a handheld control device of another endoscope.

In Example 14, the subject matter of any one or more of Examples 11-13 optionally include: wherein the controller is configured such that the control actuator can be moved from a first position relative to the working channel opening to a second position; and wherein the first pull wire tensioner is configured to tension the first pull wire in the first location and the second location.

In Example 15, the subject matter of Example 14 optionally includes wherein the anchor component is telescoping with the control component.

In Example 16, the subject matter of Example 15 optionally includes wherein the handle comprises an anti-rotation feature to prevent rotation between the anchor component and the control component.

In Example 17, the subject matter of Example 16 optionally includes wherein: the anchor component comprises: a base in which the working channel opening is disposed; and a slide extending from the base; and the control component comprises: a tube into which the slide extends; and a head to which the control actuator is mounted.

In Example 18, the subject matter of Example 17 optionally includes wherein the anti-rotation feature comprises a cross-sectional shape of the slide and tube that prevents rotation.

In Example 19, the subject matter of any one or more of Examples 11-18 optionally include wherein the anchor component and the control component are connected via a flexible handle portion.

In Example 20, the subject matter of any one or more of Examples 1-19 optionally include wherein the control actuator comprises a lever connected to the first pull wire and a second pull wire.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A controller for an endoscope, the controller comprising:
a handle;
a working channel opening connected to the handle at a first location;
a control actuator connected to the handle at a second location;
a first pull wire extending from the control actuator to the working channel opening within the handle; and
a first pull wire tensioner through which the first pull wire extends;
wherein the first pull wire tensioner is configured to remove slack from the first pull wire between the first location and the second location.

2. The controller of claim 1, wherein the control actuator is configured to pull the first pull wire to bend an insertion shaft extending from the handle, the insertion shaft including a working channel connected to the working channel opening.

3. The controller of claim 2, wherein the first pull wire tensioner comprises a curved tube.

4. The controller of claim 3, wherein the first pull wire tensioner is fabricated from a rigid material and is flexible at curvature of the curved tube.

5. The controller of claim 3, wherein the first pull wire tensioner comprises:
a first anchor fixed to the handle proximate the working channel opening; and
a second anchor fixed to the handle proximate the control actuator.

6. The controller of claim 5, wherein the first pull wire tensioner is floating within the handle along a majority of a length of the first pull wire tensioner.

7. The controller of claim 5, wherein a length along a central axis of the first pull wire tensioner between the first anchor and the second anchor is longer than a straight-line distance between the first anchor and the second anchor.

8. The controller of claim 5, wherein the curved tube is undulating.

9. The controller of claim 5, wherein the handle includes a kink between the first anchor and the second anchor.

10. The controller of claim 9, wherein the handle includes a bend between the first location and the second location such that the first pull wire is turned at least thirty degrees.

11. The controller of claim 5, wherein the handle comprises:
an anchor component connected to the working channel opening and the first anchor; and
a control component connected to the control actuator and the second anchor.

12. The controller of claim 11, wherein the anchor component and the control component are unconnected to each other and the first pull wire tensioner extends therebetween.

13. The controller of claim 12, wherein the anchor component and the control component are connected by a handheld control device of another endoscope.

14. The controller of claim 11:
wherein the controller is configured such that the control actuator can be moved from a first position relative to the working channel opening to a second position; and
wherein the first pull wire tensioner is configured to tension the first pull wire in the first location and the second location.

15. The controller of claim 14, wherein the anchor component is telescoping with the control component.

16. The controller of claim 15, wherein the handle comprises an anti-rotation feature to prevent rotation between the anchor component and the control component.

17. The controller of claim 16, wherein:
the anchor component comprises:
a base in which the working channel opening is disposed; and
a slide extending from the base; and
the control component comprises:
a tube into which the slide extends; and
a head to which the control actuator is mounted.

18. The controller of claim 17, wherein the anti-rotation feature comprises a cross-sectional shape of the slide and tube that prevents rotation.

19. The controller of claim 11, wherein the anchor component and the control component are connected via a flexible handle portion.

20. The controller of claim 1, wherein the control actuator comprises a lever connected to the first pull wire and a second pull wire.
